(19) United States
(12) Patent Application Publication
(10) Pub. No.: US 2021/0108244 A1
(43) Pub. Date: Apr. 15, 2021

(54) Method and Apparatus for Continuous Production of Sophorolipids

(71) Applicant: Locus IP Company, LLC, Solon, OH (US)

(72) Inventors: Sean FARMER, North Miami Beach, FL (US); Ken ALIBEK, Solon, OH (US); Tyler DIXON, Kent, OH (US)

(21) Appl. No.: 16/965,760

(22) PCT Filed: Jun. 27, 2019
(86) PCT No.: PCT/US2019/039445

§ 371 Date: Jul. 29, 2020

Related U.S. Application Data
(60) Provisional application No. 62/692,005, filed on Jun. 29, 2018.

(51) Int. Cl. C12P 19/44; C12M 1/00; C12M 1/12

(52) U.S. Cl. CPC C12P 19/44; C12M 47/10; C12M 37/00; C12M 23/52; C12M 29/00

(57) ABSTRACT

Systems and apparatuses for producing microbe-based compositions that can be used in the oil and gas industry, environmental cleanup, as well as for other applications are provided. More specifically, apparatuses comprising two vessels for use in large-scale production of sophorolipids are provided. An apparatus can include a fermentation vessel and a collector vessel connected directly to the fermentation vessel.

### CROSS-REFERENCE TO RELATED APPLICATION

This application claims priority to U.S. Provisional Patent Application No. 62/692,005, filed Jun. 29, 2018, which is incorporated by reference herein in its entirety.

### BACKGROUND OF THE INVENTION

Cultivation of microorganisms such as bacteria, yeast and fungi is important for the production of a wide variety of useful bio-preparations. Microorganisms play crucial roles in, for example, food industries, pharmaceuticals, agriculture, mining, environmental remediation, and waste management.

Thus, there exists an enormous potential for the use of microbes in a broad range of industries; however, an important limiting factor in commercialization of microbe-based products has been the cost per propagule density, where it is particularly expensive and unfeasible to apply microbial products to large scale operations with sufficient inoculum to see the benefits.

Two principle forms of microbe cultivation exist: submerged cultivation and surface cultivation. Bacteria, yeasts and fungi can all be grown using either the surface or submerged cultivation methods. Both cultivation methods require a nutrient medium for the growth of the microorganisms. The nutrient medium, which can either be in a liquid or a solid form, typically includes a carbon source, a nitrogen source, salts and appropriate additional nutrients and microelements. The pH and oxygen levels are maintained at values suitable for a given microorganism.

Microbes have the potential to play highly beneficial roles in, for example, the oil and agriculture industries, if only they could be made more readily available and, preferably, in a more active form.

Oil and natural gas are obtained by drilling into the earth's surface using what is generically referred to as a drilling rig. A well or borehole begins by drilling a large diameter hole (e.g., 24-36 inches in diameter) into the ground using a drill bit. The drill bit is attached to a drill pipe, which is rotated by the drilling rig. The drilling rig generally continues to drill a large hole until the drill bit passes beneath the water table. Next, a metal liner (or casing) is placed in the large diameter hole and cement is pumped through the inside of the liner. When the cement reaches the bottom of the liner, it flows upward, filling the void between the liner and the surrounding formation, isolating the water table and protecting it from whatever drilling fluids are pumped down the hole in subsequent steps.

After the first casing is cemented in, a medium sized bit can be used to drill deeper into the subterranean formation. There are generally one or more stopping points where the drill bit is removed, followed by a smaller casing liner and cement. This process is repeated until the well is completed.

During the drilling process, drilling fluids are pumped through the drill pipe and out of the drill bit. This fluid then flows back up in the space between the drill pipe and the formation or casing. The drilling fluid removes drill cuttings, balances downhole pressures, lubricates the borehole, and also works to clean the borehole of friction-causing substances.

After the well is drilled, a production liner (or casing) is generally set and the well is then perforated (e.g., explosives are used to puncture the production liner at specific points in the oil bearing formation). Oil then begins to flow out of the well, either under the natural pressure of the formation or by using pressure that is induced via mechanical equipment, water flooding, or other means. As the crude oil flows through the well, substances in the crude oil often collect on the surfaces of the production liners, causing reduction in flow, and sometimes even stopping production all together.

A variety of different chemicals and equipment are utilized to prevent and remediate this issue, but there is a need for improved products and methods. In particular, there is a need for products and methods that are more environmentally friendly, less toxic, and have improved effectiveness.

In the agriculture industry, farmers have relied heavily on the use of synthetic chemicals and chemical fertilizers to boost yields and protect crops against pathogens, pests, and disease; however, when overused or improperly applied, these substances can be air and water pollutants through runoff, leaching and evaporation. Even when properly used, the over-dependence and long-term use of certain chemical fertilizers and pesticides deleteriously alters soil ecosystems, reduces stress tolerance, increases pest resistance, and impedes plant and animal growth and vitality.

Mounting regulatory mandates governing the availability and use of chemicals, and consumer demands for residue free, sustainably-grown food produced with minimal harm to the environment, are impacting the industry and causing an evolution of thought regarding how to address the myriad of challenges. The demand for safer pesticides and alternate pest control strategies is increasing. While wholesale elimination of chemicals is not feasible at this time, farmers are increasingly embracing the use of biological measures as viable components of Integrated Nutrient Management and Integrated Pest Management programs.

For example, in recent years, biological control of nematodes has caught great interest. This method utilizes biological agents as pesticides, such as live microbes, bio-products derived from these microbes, and combinations thereof. These biological pesticides have important advantages over other conventional pesticides. For example, they are less harmful compared to the conventional chemical pesticides. They are more efficient and specific. They often biodegrade quickly, leading to less environmental pollution.

The use of biopesticides and other biological agents has been greatly limited by difficulties in production, transportation, administration, pricing and efficacy. For example, many microbes are difficult to grow and subsequently deploy to agricultural and forestry production systems in sufficient quantities to be useful. This problem is exacerbated by losses in viability and/or activity due to processing, formulating, storage, and stabilizing prior to distribution. Furthermore, once applied, biological products may not thrive for any number of reasons including, for example, insufficient initial cell densities, the inability to compete effectively with the existing microflora at a particular location, and being introduced to soil and/or other environmental conditions in which the microbe cannot flourish or even survive.

Microbe-based compositions could help resolve some of the aforementioned issues faced by the agriculture industry, the oil and gas industry, as well as many others. Thus, there is a need for more efficient cultivation methods for mass production of microorganisms and microbial metabolites.

Sophorolipids (SLP) are biosurfactants belonging to the class of glycolipids produced by many non-pathogenic yeasts. SLPs are one of the best known biosurfactants with many advantages over synthetic surfactants. SLPs can be used in agriculture, food preservation, biomedicine, cosmetics, bioremediation, crude oil recovery, removal of heavy metals, nanotechnology (e.g., formation of nanoparticles), desludging of various liquid wastes, and making various household products. However, significant obstacles exist in production of SLPs, including the relatively high cost of manufacturing and the difficulty of large-scale production.

### BRIEF SUMMARY OF THE INVENTION

The present invention provides materials, methods and systems for producing microbe-based compositions that can be used in the oil and gas industry, agriculture, health care and environmental cleanup, as well as for a variety of other applications. Specifically, the subject invention provides materials, methods and systems for efficient cultivation of microorganisms and production of microbial growth by-products.

Embodiments of the present invention provide novel apparatuses and methods for continuous, and relatively inexpensive, production of sophorolipids (SLP), including SLP-based Starmerella bombicola, that can be used in, for example, the oil industry, agriculture, aquaculture, mining, waste treatment, and bioremediation. An apparatus can include two vessels capable of producing large amounts of SLP. The two vessels comprise a fermentation vessel and a collector vessel, which may be connected to each other. Alternatively, the vessels can be at different locations, and cells and/or other contents from the fermentation vessel can be freeze dried and transported to the collector vessel at a different location, for example if the collector vessel is the site where SLP will be used and the fermentation vessel is not (e.g., fermentation vessel is centrally located). In this alternative case, even if the fermentation vessel is located off site, the SLP can advantageously be located at the site where it will be used, so that it need not be transported after it is ready for use.

Embodiments of the present invention are concerned with non-stop/continuous fermentation. In the fermentation vessel, the culture is moving, such that SLP and cells do not settle. In the collection vessel, the cells and medium can be static, such that the volume is kept constant or approximately constant by removing volume (e.g., leftover nutrients, metabolic end products, and microbes) at the same rate that volume (e.g., culture cells) is added. The SLP can settle to the bottom in the collector vessel, and when there is little or no disturbance, it precipitates.

In one embodiment, a reactor apparatus can include a fermentation vessel and a collector vessel connected to the fermentation vessel. The reactor vessel grows culture by fermentation, and the culture can be used to produce SLP. The culture can be, for example, Starmerella bombicola culture. The collector vessel serves as a collector of a portion of the culture and for precipitation of accumulated SLP. The SLP can settle to the bottom of the collector vessel and precipitate. Some or all of the precipitated SLP product can be collected from the collector vessel. For example, gravity can be used to drain the product out of the collector vessel for collection or a pump (e.g., a peristaltic pump) can be used to pump the product out for collection.

The reactor apparatus can be self-sterilizing. Once set up and cleaned initially, it does not have to be cleaned. SLP provides self-treatment of sepsis, even at low concentrations. This saves time and cost of sterilization.

In one embodiment, the subject invention provides methods of cultivating microorganisms without contamination using the subject system. In certain embodiments, the methods of cultivation comprise adding a culture medium comprising water and nutrient components to the subject systems using, for example, a peristaltic pump; inoculating the system with a viable microorganism; and optionally, adding an antimicrobial agent to the culture medium. The antimicrobial agent can be, for example, an antibiotic or a sophorolipid.

In one embodiment, the subject invention further provides a composition comprising at least one type of microorganism and/or at least one microbial metabolite produced by the microorganism that has been grown using the subject fermentation system. The microorganisms in the composition may be in an active or inactive form. The composition may also be in a dried form or a liquid form.

Advantageously, the methods and equipment of the subject invention reduce the capital and labor costs of producing microorganisms and their metabolites on a large scale. Furthermore, the cultivation process of the subject invention reduces or eliminates the need to concentrate organisms after completing cultivation. The subject invention provides a cultivation method that not only substantially increases the yield of microbial products per unit of nutrient medium but simplifies production and facilitates portability.

Portability can result in significant cost savings as microbe-based compositions can be produced at, or near, the site of intended use. This means that the final composition can be manufactured on-site using locally-sourced materials if desired, thereby reducing shipping costs. Furthermore, the compositions can include viable microbes at the time of application, which can increase product effectiveness.

Thus, in certain embodiments, the systems and apparatuses of the subject invention harness the power of naturally-occurring local microorganisms and their metabolic by-products. Use of local microbial populations can be advantageous in settings including, but not limited to, agriculture, environmental remediation (such as in the case of an oil spill), animal husbandry, aquaculture, forestry, pasture management, turf management, horticultural ornamental production, waste disposal and treatment, mining, oil recovery, and human health, including in remote locations.

Compositions produced by the present invention can also be used in a wide variety of petroleum industry applications, such as microbially-enhanced oil recovery. These applications include, but are not limited to, enhancement of crude oil recovery; stimulation of oil and gas wells (to improve the flow of oil into the well bore); removal of contaminants and/or obstructions such as paraffins, asphaltenes and scale from equipment such as rods, tubing, liners, tanks and pumps; prevention of the corrosion of oil and gas production and transportation equipment; reduction of H₂S concentration in crude oil and natural gas; reduction in viscosity of crude oil; upgradation of heavy crude oils and asphaltenes into lighter hydrocarbon fractions; cleaning of tanks, flowlines and pipelines; enhancing the mobility of oil during water flooding though selective and non-selective plugging; and fracturing fluids.

When used in oil and gas applications, the systems and apparatuses of the present invention can be used to lower the cost of microbial-based oilfield compositions and can be used in combination with other chemical enhancers, such as polymers, solvents, fracking sand and beads, emulsifiers, surfactants, and other materials known in the art.

### BRIEF DESCRIPTION OF THE DRAWINGS

FIG. 1 shows an image of a two-vessel reactor vessel according to an embodiment of the present invention.

### DETAILED DESCRIPTION OF THE INVENTION

The present invention provides materials, methods and systems for producing microbe-based compositions that can be used in the oil and gas industry, agriculture, health care and environmental cleanup, as well as for a variety of other applications. Specifically, the subject invention provides materials, methods and systems for efficient cultivation of microorganisms and production of microbial growth by-products.

Embodiments of the present invention provide novel, low-cost fermentation methods and systems. More specifically, the present invention provides biological reactors (also referred to herein as “systems,” “fermentation systems,” “reactor systems,” and/or “units”) for fermenting a wide variety of, for example, bio level 1 microorganisms with very high cell densities. In specific embodiments, the systems are used to grow yeast- and/or other microbe-based compositions. In certain specific embodiments, the systems can be used for the production of Starmerella bombicola yeast compositions.

The systems can be used to grow yeast, fungi and bacteria. In certain embodiments, the systems can be used for the production of yeast-based compositions, including, for example, compositions comprising Starmerella bombicola, Wickerhamomyces anomalus, and/or Pseudozyma aphidis yeast. In some embodiments, the systems can be used for the production of bacteria-based compositions, including, for example, compositions comprising Bacillus subtilis and/or Bacillus licheniformis.

In a preferred embodiment wherein yeasts are cultured, the resulting composition can have one or more of the following advantageous properties: high concentrations of mannoprotein and beta-glucan as part of the yeasts' cell wall; and the presence of biosurfactants and other microbial metabolites (e.g., lactic acid and ethanol, etc.) in the culture.

### Selected Definitions

As used herein, reference to a “microbe-based composition” means a composition that comprises components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of propagule, or a mixture of these. The microbes may be planktonic or in a biofilm form, or a mixture of both. The by-products of growth may be, for example, metabolites, cell membrane components, expressed proteins, and/or other cellular components. The microbes may be intact or lysed. In preferred embodiments, the microbes are present, with broth in which they were grown, in the microbe-based composition. The cells may be present at, for example, a concentration of at least 1×10⁴, 1×10⁵, 1×10⁶, 1×10⁷, 1×10⁸, 1×10⁹, 1×10¹⁰, or 1×10¹¹ or more CFU per ml of composition.

The subject invention further provides “microbe-based products,” which are products that are to be applied in practice to achieve a desired result. The microbe-based product can be simply the microbe-based composition harvested from the microbe cultivation process. Alternatively, the microbe-based product may comprise further ingredients that have been added. These additional ingredients can include, for example, stabilizers, buffers, appropriate carriers, such as water, salt solutions, or any other appropriate carrier, added nutrients to support further microbial growth, non-nutrient growth enhancers, such as plant hormones, and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, “harvested” refers to removing some or all of the microbe-based composition from a growth vessel.

As used herein, a “biofilm” is a complex aggregate of microorganisms, such as bacteria, wherein the cells adhere to each other and/or to a surface using an extracellular polysaccharide matrix. The cells in biofilms are physiologically distinct from planktonic cells of the same organism, which are single cells that can float or swim in liquid medium.

As used herein, the term “control” used in reference to the activity produced by the subject microorganisms extends to the act of killing, disabling or immobilizing pests or otherwise rendering the pests substantially incapable of causing harm.

As used herein, an “isolated” or “purified” nucleic acid molecule, polynucleotide, polypeptide, protein or organic compound such as a small molecule (e.g., those described below), is substantially free of other compounds, such as cellular material, with which it is associated in nature. As used herein, reference to “isolated” in the context of a microbial strain means that the strain is removed from the environment in which it exists in nature. Thus, the isolated strain may exist as, for example, a biologically pure culture, or as spores (or other forms of the strain) in association with a carrier.

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state.

A “metabolite” refers to any substance produced by metabolism or a substance necessary for taking part in a particular metabolic process. A metabolite can be an organic compound that is a starting material, an intermediate in, or an end product of metabolism. Examples of metabolites include, but are not limited to, enzymes, acids, solvents, alcohols, proteins, vitamins, minerals, microelements, amino acids, and biosurfactants.

As used herein, “surfactant” refers to a compound that lowers the surface tension (or interfacial tension) between two liquids or between a liquid and a solid. Surfactants act as, e.g., detergents, wetting agents, emulsifiers, foaming agents, and dispersants. A “biosurfactant” is a surfactant produced by a living organism.

### Reactor System Design and Operation

Embodiments of the present invention provide novel apparatuses and methods for continuous, and relatively inexpensive, production of sophorolipids (SLP), including SLP-based Starmerella bombicola, that can be used in, for example, the oil industry, agriculture, aquaculture, mining, waste treatment, and bioremediation. An apparatus can include two vessels capable of producing large amounts of SLP. The two vessels comprise a fermentation vessel and a collector vessel, which may be connected to each other. Alternatively, the vessels can be at different locations, and cells and/or other contents from the fermentation vessel can be freeze dried and transported to the collector vessel at a different location, for example if the collector vessel is the site where SLP will be used and the fermentation vessel is not (e.g., fermentation vessel is centrally located). In this alternative case, even if the fermentation vessel is located off site, the SLP can advantageously be located at the site where it will be used, so that it need not be transported after it is ready for use.

Embodiments of the present invention are concerned with non-stop/continuous fermentation. In the fermentation vessel, the culture is moving, such that SLP and cells do not settle. In the collection vessel, the cells and medium can be static, such that the volume is kept constant or approximately constant by removing volume (e.g., leftover nutrients, metabolic end products, and microbes) at the same rate that volume (e.g., culture cells) is added. The SLP can settle to the bottom in the collector vessel, and when there is little or no disturbance, it precipitates.

In one embodiment, a reactor apparatus can include a fermentation vessel and a collector vessel connected to the fermentation vessel. The reactor vessel grows culture by fermentation, and the culture can be used to produce SLP. The culture can be, for example, Starmerella bombicola culture. The collector vessel serves as a collector of a portion of the culture and for precipitation of accumulated SLP. The SLP can settle to the bottom of the collector vessel and precipitate. Some or all of the precipitated SLP product can be collected from the collector vessel. For example, gravity can be used to drain the product out of the collector vessel for collection or a pump (e.g., a peristaltic pump) can be used to pump the product out for collection. In a further embodiment, the collector vessel can have a sight glass to allow visual observation from outside the vessel of SLP accumulation.

The reactor system/apparatus can be self-sterilizing. Once set up and cleaned initially, it does not have to be cleaned. SLP provides self-treatment of sepsis, even at low concentrations. This saves time and cost of sterilization.

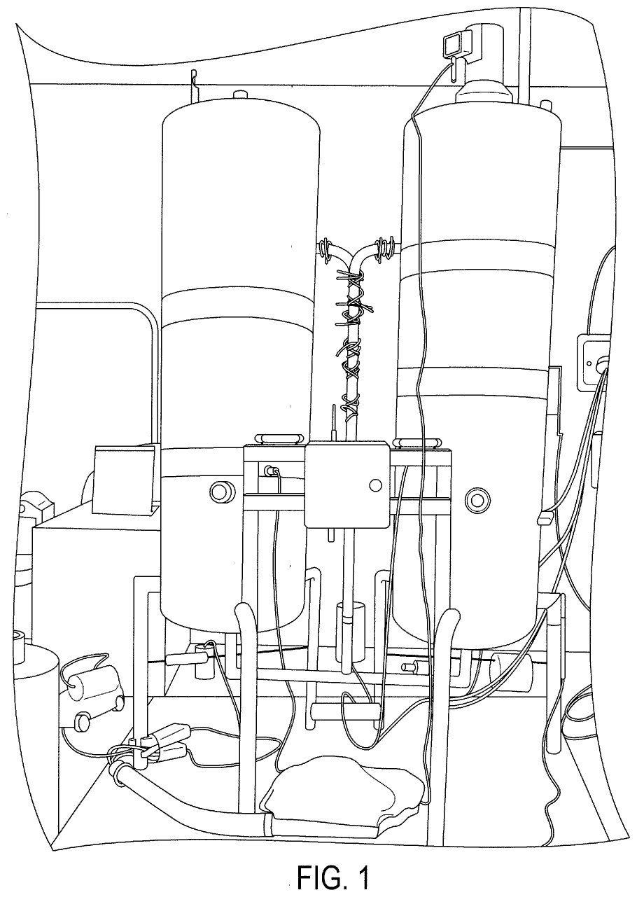

FIG. 1 shows an image of a two-vessel reactor apparatus. Referring to FIG. 1, the apparatus can include a fermentation vessel and a collector vessel connected to the fermentation vessel. The collector vessel serves as a collector of a portion of the culture from the fermentation vessel and for precipitation of accumulated SLP, which can then be removed/collected from the collection vessel. The two vessels can be connected by any suitable means known in the art, for example by one or more pipes. The vessels can be positioned relatively close to each other to minimize the length of pipe or other connection means between them. A total height of the fermentation vessel can be the same as that of the collector vessel, though embodiments are not limited thereto. Each vessel can be, for example a metal vessel, though embodiments are not limited thereto. Each vessel can be supported by a frame (e.g., a metal frame), such that it is easier to access and to view the inside through a sight glass (if present). Large amounts of SLP can be produced by this reactor apparatus/system.

In one embodiment, the fermentation vessel is supplemented with an impeller, a jacket, or both to ensure efficient parameters of oxygen saturation and temperature range.

In one embodiment, the culture from the collector vessel, after SLP is collected from the collector vessel, is returned to the fermentation vessel to continue fermentation. For example, the culture from the collector vessel can be pumped back to the fermentation vessel. This culture can be returned through the same or a different connection between the two vessels than that used to supply the collector vessel with the culture from the fermentation vessel. In this context, the collector vessel can also be referred to as the collector/feeding vessel. In a further embodiment, the culture in the collector/feeding vessel, before being provided back to the fermentation vessel, is supplemented with two carbon sources, including a sugar source (e.g., glucose, sucrose, etc.) and a fatty acid source (e.g., canola oil, soybean oil, etc.).

In one embodiment, two carbon sources, including a sugar source (e.g., glucose, sucrose, etc.) and a fatty acid source (e.g., canola oil, soybean oil, etc.), are added directly to the fermentation vessel. This can be done during the initial fermentation process, during a continued fermentation process after culture is returned from the collector vessel, or during both.

In one embodiment, when the reactor apparatus/system is run, fermentation is initially allowed to proceed for multiple days (e.g., for a period of time in a range of from 4 to 9 days, or more specifically from 4 to 5 days) to allow for SLP production. The SLP can settle to the bottom of the collector vessel and precipitate. The SLP can then be sampled from the collector vessel, and the concentration and/or total amount of SLP can be measured. If a satisfactory amount of SLP has accumulated for the intended end use (for example, if one third or approximately one third of the collector vessel contents (e.g., by volume) is SLP), SLP can then be collected regularly (e.g., daily) from the collector vessel. For example, gravity can be used to drain the product out of the collector vessel for collection or a pump (e.g., a peristaltic pump) can be used to pump the product out for collection. In a further embodiment, the collector vessel can have a sight glass to allow visual observation from outside the vessel of SLP accumulation.

In one embodiment, the apparatus can include at least one additional vessel containing one or more carbon sources. For example, one additional vessel can be included and can contain a sugar source (e.g., glucose, sucrose, etc.) and a fatty acid source (e.g., canola oil, soybean oil, etc.) and can be connected directly to the fermentation vessel, the collector vessel, or both. Alternatively, two additional vessels can be included, one containing a sugar source (e.g., glucose, sucrose, etc.) and the other containing a fatty acid source (e.g., canola oil, soybean oil, etc.). The two additional vessels can be connected directly to the fermentation vessel, the collector vessel, or both.

In one embodiment, the fermentation vessel and the collector vessel are not connected. Starmerella bombicola yeast are grown in the fermentation vessel. When the yeast are growing and multiplying, they are not producing much, if any, SLP. The yeast can be grown in a sterilized nutrient medium comprising a nitrogen source, a protein source (e.g., yeast extract), urea, glucose and/or sucrose at a concentration of up to 100 grams per liter of culture, and an oil/fat. Different oils/fats having different lengths of fatty acid chains can be used. Soybean and canola oil have more oleic acid, which is a component for biosurfactant production. Preferably, the ratio of sugar and oil during fermentation is 1:1. The initial pH is in a range of from 5.5 to 6.0, and the fermentation vessel is inoculated with the yeast and fermentation occurs for multiple days (e.g., for a period of time in a range of from 4 to 9 days, or more specifically from 5 to 9 days), or until the yeast becomes nitrogen-exhausted. These resulting cells are the biomass. The cells can also optionally be immobilized in alginate beads, or frozen, dried, or freeze-dried. The biomass can be collected (e.g., by centrifuge, filter, sedimentation, etc.) and then taken to a collector vessel (e.g., a column). The collector vessel may have a mixer, though it is not required. To minimize the chances of the cells multiplying, no nutrient medium is provided to the collector vessel, though water can be provided. The yeast can be activated in the vessel by adding a limited amount of a nitrogen source or other known activator. The amount of nitrogen should be less than the amount used in the fermentation vessel (to minimize the chances of cell multiplication). SLPs include the sugar sophorose (disaccharide) linked to a long-chain hydroxyl fatty acid. When activated, S. bombicola excretes enzymes that convert oleic acid and glucose to SLP. As soon as the cells are active (in SLP production), carbon sources can be added to the collector vessel, and the carbon sources can include a sugar and an oil for biosurfactant production; the ratio of sugar:oil can be 2:1. For example, the sugar (e.g., glucose, sucrose, etc.) can be added at a concentration of 10-150 g/L, and the oil can be added at an concentration of 10-100 mL.

The temperature of the collector vessel can be kept in a range of from 20° C. to 30° C., preferably from 22° C. to 25° C., and the pH can be 3 to 4, preferably 3.5. The dissolved oxygen can be from 5% to 30% of ambient air, preferably from 10% to 30% of ambient air. As SLP is produced in the collector vessel, it sinks to the bottom. At this point, a second portion of sugar/oil can be added. As soon as SLP settles, some of the yeast culture and SLP can be collected and placed back in the first vessel to keep the process going. To keep SLP production going, sugar and oil can consistently be added into the second vessel. Purified fatty acids (oleic, linoleic, etc.) can be added and/or glucose/sucrose can be replaced with disaccharides to increase the speed of production and decrease energy required. This is because it reduces the steps of converting the raw components into SLP.

The process discussed in the previous paragraph allows for the potential use of different locations and facilities. For example, the fermentation vessel and collector vessel can be located in different places. This is advantageous because the cells can be frozen, dried, or freeze dried and transported to implement SLP production in a different location from where the cells are produced (for example, if the cells are produced at a site different from where the SLP will be used). Advantageously, the process does not require a significant amount of cells to produce high concentrations of SLP. Further, sterilization/sanitization of the collector vessel is not required because the cells aren't growing and because SLP is present. However, antibiotics or other control agents can be used if desired. The process can be implemented as a batch or quasi-continuous process. For example, SLP and yeast from the collector vessel can be collected and provided back to a fermentation vessel (either the original fermentation vessel that was already used or another reactor altogether). After two or three cycles of SLP synthesis in the collector vessel, a nitrogen source can be added to the collector vessel to allow cells to multiply one or two times and keep cells in the system.

The system/apparatus can also be adapted to ensure maintaining an appropriate fermentation temperature. For example, the outside of the system can be reflective to avoid raising the system temperature during the day if being operated outdoors. The system can also be insulated so the fermentation process can remain at appropriate temperatures in low temperature environments. Any of the insulating materials known in the art can be applied including fiberglass, silica aerogel, ceramic fiber insulation, etc. The insulation can surround any and/or all of the tubes and/or tanks of the system.

A thermometer can be included, which can be a manual or automatic thermometer. An automatic thermometer can manage the heat and cooling sources appropriately to control the temperature throughout the fermentation process. The desired temperatures can be programmed on-site or pre-programmed before the system is delivered to the fermentation site. The temperature measurements can then be used to automatically control the temperature control systems that are discussed above.

The pH adjustment can be accomplished by automatic means or it can be done manually. The automatic pH adjustment can include a pH probe and an electronic device to dispense pH adjustment substances appropriately, depending on the pH measurements. The pH can be set to a specific number by a user or can be pre-programmed to change the pH accordingly throughout the fermentation process. If the pH adjustment is to be done manually, pH measurement tools known in the art can be included with the system for manual testing.

A computer system for measuring and adjusting of pH and temperature can be used to monitor and control fermentation parameters for the reactor apparatus. The computer can be connected to a thermometer and a pH probe, for example. In addition to monitoring and controlling temperature and pH, each vessel may also have the capability for monitoring and controlling, for example, dissolved oxygen, agitation, foaming, purity of microbial cultures, production of desired metabolites and the like. The systems/apparatuses can further be adapted for remote monitoring of these parameters, for example with a tablet, smart phone, or other mobile computing device capable of sending and receiving data wirelessly.

In a further embodiment, the vessels may also be able to monitor the growth of microorganisms inside the vessel (e.g., measurement of cell number and growth phases).

Alternatively, a daily sample may be taken from the vessel and subjected to enumeration by techniques known in the art, such as dilution plating technique. Dilution plating is a simple technique used to estimate the number of bacteria in a sample. The technique can also provide an index by which different environments or treatments can be compared.

The system/apparatus can include one or more frames for supporting the apparatus components (including the vessels, pipes, pumps, impellers, jackets, etc.). The system/apparatus can include wheels for moving the apparatus, as well as handles for steering, pushing and pulling when maneuvering the apparatus.

The system/apparatus can be configured on the back of one or more truck trailers and/or semi-trailers. That is, the system/apparatus can be designed to be portable (i.e., the system can be suitable for being transported on a pickup truck, a flatbed trailer, or a semi-trailer).

### Microorganisms

The microorganisms produced according to the subject invention can be, for example, bacteria, yeasts and/or fungi. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In one embodiment, the microorganism is a yeast or fungus. Yeast and fungus species suitable for use according to the current invention, include Acaulospora, Aspergillus, Aureobasidium (e.g., A. pullulans), Blakeslea, Candida (e.g., C. albicans, C. apicola, C. batistae, C. bombicola, C. floricola, C. kuoi, C. riodocensis, C. stellate), Debaryomyces (e.g., D. hansenii), Entomophthora, Glomus (e.g., G. mosseae), Hanseniaspora (e.g., H. uvarum), Hansenula, Issatchenkia, Kluyveromyces, Lentinula edodes, Mortierella, Mucor (e.g., M piriformis), Penicillium, Phythium, Phycomyces, Pichia (e.g., P. anomala, P. guielliermondii, P. occidentalis, P. kudriavzevii), Pseudozyma (e.g., P. aphidis), Rhizopus, Rhodotorula (e.g., R. bogoriensis); Saccharomyces (S. cerevisiae, S. boulardii sequela, S. torula), Starmerella (e.g., S. bombicola), Torulopsis, Thraustochytrium, Trichoderma (e.g., T reesei, T. harzianurn, T. virens), Ustilago (e.g., U maydis), Wickerhamiella (e.g., W. domericqiae), Wickerhamomyces (e.g., W. anomalus), Williopsis, Zygosaccharomyces.

In one embodiment, the microorganism is a yeast known as a “killer yeast.” As used herein, “killer yeast” means a strain of yeast characterized by its secretion of toxic proteins or glycoproteins, to which the strain itself is immune. Killer yeasts can include, but are not limited to species of, for example, Candida (e.g., C. nodaensis), Cryptococcus, Debaryomyces (e.g., D. hansenii), Hanseniaspora, (e.g., H. uvarum), Hansenula, Kluyveromyces (e.g., K. phaffii), Pichia (e.g., P. anomala, P. guielliermondii, P. occidentalis, P. kudriavzevii), Saccharomyces (e.g., S. cerevisiae), Torulopsis, Ustilago (e.g., U. maydis), Wickerhamomyces (e.g., W. anomalus), Williopsis (e.g., W. mrakii), Zygosaccharomyces (e.g., Z. bailii), and others.

In one embodiment, the microbe is a killer yeast, such as a Pichia yeast selected from Pichia anomala (Wickerhamomyces anomalus), Pichia guilliermondii, and Pichia kudriavzevii. Pichia anomala, in particular, is an effective producer of various solvents, enzymes, killer toxins, as well as glycolipid biosurfactants.

In one embodiment, the microbial strain is chosen from the Starmerella clade. A culture of a Starmerella microbe useful according to the present invention, Starmerella bombicola, can be obtained from the American Type Culture Collection (ATCC), 10801 University Blvd., Manassas, Va. 20110-2209 USA. The deposit has been assigned accession number ATCC No. 22214 by the depository.

The system/apparatus can also utilize one or more strains of yeast capable of enhancing oil recovery and performing paraffin degradation, e.g., Starmerella (Candida) bombicola, Candida apicola, Candida batistae, Candida floricola, Candida riodocensis, Candida stellate, Candida kuoi, Candida sp. NRRL Y-27208, Rhodotorula bogoriensis sp., Wickerhamiella domericqiae, as well as any other sophorolipid-producing strains of the Starmerella clade. In a specific embodiment, the yeast strain is ATCC 22214 and mutants thereof.

In one embodiment, the microbe is a strain of Pseudozyma aphidis. This microbe is an effective producer of mannosylerythritol lipid biosurfactants.

In one embodiment, the microorganisms are bacteria, including Gram-positive and Gram-negative bacteria, as well as some archaea. The bacteria may be, spore-forming, or not. The bacteria may be motile or sessile. The bacteria may be anaerobic, aerobic, microaerophilic, facultative anaerobes and/or obligate aerobes. Bacteria species suitable for use according to the present invention include, for example, Acinetobacter (e.g., A. calcoaceticus, A. venetianus); Agrobacterium (e.g., A. radiobacter), Azotobacter (A. vinelandii, A. chroococcum), Azospirillum (e.g., A. brasiliensis), Bacillus (e.g., B. amyloliquefaciens, B. firmus, B. laterosporus, B. licheniformis, B. megaterium, B. mucilaginosus, B. subtilis, B. coagulans GBI-30 (BC30)), Chloroblaceae spp., Dyadobacter fermenters, Frankia spp., Frateuria (e.g., F. aurantia), Klebsiella spp., Microbacterium (e.g., M. laevaniformans), Pantoea (e.g., P. agglomerans), Pseudomonas (e.g., P. aeruginosa, P. chlororaphis, P. chlororaphis subsp. aureofaciens (Kluyver), P. putida), Rhizobium spp., Rhodospirillum (e.g., R. rubrum), Sphingomonas (e.g., S. paucimobilis), and/or Xanthomonas spp.

In one embodiment, the microorganism is a strain of B. subtilis, such as, for example, B. subtilis var. lotuses B1 or B2, which are effective producers of, for example, surfactin and other biosurfactants, as well as biopolymers. This specification incorporates by reference International Publication No. WO 2017/044953 A1 to the extent it is consistent with the teachings disclosed herein. In another embodiment, the microorganism is a strain of Bacillus licheniformis, which is an effective producer of biosurfactants as well as biopolymers, such as levan.

Other microbial strains including, for example, strains capable of accumulating significant amounts of, for example, glycolipid-biosurfactants, can be used in accordance with the present invention. Other microbial by-products useful according to the present invention include mannoprotein, beta-glucan and other metabolites that have bio-emulsifying and surface/interfacial tension-reducing properties.

In one embodiment, a single type of microbe is grown in a vessel. In alternative embodiments, multiple microbes, which can be grown together without deleterious effects on growth or the resulting product, can be grown in a single vessel. There may be, for example, 2 to 3 or more different microbes grown in a single vessel at the same time.

### Methods of Cultivation Using the Subject Fermentation Systems

In one embodiment, the present invention provides methods of cultivating microorganisms without contamination using the present system/apparatus. In certain embodiments, the methods of cultivation comprise adding a culture medium comprising water and nutrient components to the subject systems using, for example, a peristaltic pump; inoculating the system with a viable microorganism; and optionally, adding an antimicrobial agent to the culture medium. The antimicrobial agent can be, for example, an antibiotic or a sophorolipid.

In one embodiment, the present invention further provides a composition comprising at least one type of microorganism and/or at least one microbial metabolite produced by the microorganism that has been grown using the subject fermentation system. The microorganisms in the composition may be in an active or inactive form. The composition may also be in a dried form or a liquid form. In one embodiment, the composition comprises the microbial metabolite but not the microorganism, where the microorganism or microorganisms are separated from the metabolite(s) and/or other culture medium components.

Prior to microbe growth, the vessels of the apparatus may be disinfected or sterilized. In one embodiment, fermentation medium, air, and equipment used in the method and cultivation process are sterilized. The cultivation equipment such as the reactor/vessel may be separated from, but connected to, a sterilizing unit, e.g., an autoclave. The cultivation equipment may also have a sterilizing unit that sterilizes in situ before starting the inoculation, e.g., by using a steamer. The air can be sterilized by methods know in the art. For example, the ambient air can pass through at least one filter before being supplemented into the vessel. In other embodiments, the medium may be pasteurized or optionally no heat at all added, where the use of low water activity and low pH may be exploited to control unwanted bacterial growth.

Advantageously, the method and equipment of the present invention reduce the capital and labor costs of producing microorganisms and their metabolites on a large scale. Furthermore, the cultivation process of the present invention reduces or eliminates the need to concentrate organisms after completing cultivation. The present invention provides a cultivation method that not only substantially increases the yield of microbial products per unit of nutrient medium but simplifies production and facilitates portability.

Portability can result in significant cost savings as microbe-based compositions can be produced at, or near, the site of intended use. This means that the final composition can be manufactured on-site using locally-sourced materials if desired, thereby reducing shipping costs. Furthermore, the compositions can include viable microbes at the time of application, which can increase product effectiveness.

Thus, in certain embodiments, the systems/apparatuses of the present invention harness the power of naturally-occurring local microorganisms and their metabolic by-products. Use of local microbial populations can be advantageous in settings including, but not limited to, environmental remediation (such as in the case of an oil spill), animal husbandry, aquaculture, forestry, pasture management, turf management, horticultural ornamental production, waste disposal and treatment, mining, oil recovery, and human health, including in remote locations.

The present invention provides methods and systems/apparatuses for the efficient production of microbes using novel biological reactors. The system can include all of the materials necessary for the fermentation (or cultivation) process, including, for example, equipment, sterilization supplies, and culture medium components, although it is expected that freshwater could be supplied from a local source and sterilized according to the subject methods.

In one embodiment, the system/apparatus is provided with an inoculum of viable microbes. Preferably, the microbes are biochemical-producing microbes, capable of accumulating, for example, biosurfactants, enzymes, solvents, biopolymers, acids, and/or other useful metabolites. In particularly preferred embodiments, the microorganisms are biochemical-producing yeast (including killer yeasts), fungi, and/or bacteria, including without limitation those listed herein.

In one embodiment, the system/apparatus is provided with a culture medium. The medium can include nutrient sources, for example, a carbon source, a lipid source, a nitrogen source, and/or a micronutrient source. Each of the carbon source, lipid source, nitrogen source, and/or micronutrient source can be provided in an individual package that can be added to the reactor at appropriate times during the fermentation process. Each of the packages can include several sub-packages that can be added at specific points (e.g., when yeast, pH, and/or nutrient levels go above or below a specific concentration) or times (e.g., after 10 hours, 20 hours, 30 hours, 40 hours, etc.) during the fermentation process.

Before fermentation the tank can be washed with a hydrogen peroxide solution (e.g., from 2.0% to 4.0% hydrogen peroxide; this can be done before or after a hot water rinse at, e.g., 80-90° C.) to prevent contamination. In addition, or in the alternative, the tank can be washed with a commercial disinfectant, a bleach solution and/or a hot water or steam rinse. The system/apparatus can come with concentrated forms of the bleach and hydrogen peroxide, which can later be diluted at the fermentation site before use. For example, the hydrogen peroxide can be provided in concentrated form and be diluted to formulate 2.0% to 4.0% hydrogen peroxide (by weight or volume) for pre-rinse decontamination.

In a specific embodiment, the method of cultivation comprises sterilizing the subject fermentation reactors prior to fermentation. The internal surfaces of the reactor (including, e.g., tanks, ports, spargers and mixing systems) can first be washed with a commercial disinfectant; then fogged (or sprayed with a highly dispersed spray system) with 2% to 4% hydrogen peroxide, preferably 3% hydrogen peroxide; and finally steamed with a portable steamer at a temperature of about 105° C. to about 110° C., or greater.

The culture medium components (e.g., the carbon source, water, lipid source, micronutrients, etc.) can also be sterilized. This can be achieved using temperature decontamination and/or hydrogen peroxide decontamination (potentially followed by neutralizing the hydrogen peroxide using an acid such as HCl, H₂SO₄, etc.).

In a specific embodiment, the water used in the culture medium is UV sterilized using an in-line UV water sterilizer and filtered using, for example, a 0.1-micron water filter. In another embodiment, all nutritional and other medium components can be autoclaved prior to fermentation.

To further prevent contamination, the culture medium of the system may comprise additional acids, antibiotics, and/or antimicrobials, added before, and/or during the cultivation process. The one or more antimicrobial substances can include, e.g., streptomycin, oxytetracycline, sophorolipids, and rhamno lipid s.

Inoculation can take place in any and/or all of the reactor tanks, at which point the inoculum is mixed using through the tubing systems. Total fermentation times can range from 10 to 200 hours, preferably from 20 to 180 hours.

The fermenting temperature utilized in the subject system/apparatus and methods can be, for example, from about 25 to 40° C., although the process may operate outside of this range. In one embodiment, the method for cultivation of microorganisms is carried out at about 5° to about 100° C., preferably, 15° to 60° C., more preferably, 25 to 50° C. In a further embodiment, the cultivation may be carried out continuously at a constant temperature. In another embodiment, the cultivation may be subject to changing temperatures.

The pH of the medium should be suitable for the microorganism of interest. Buffering salts, and pH regulators, such as carbonates and phosphates, may be used to stabilize pH near an optimum value. When metal ions are present in high concentrations, use of a chelating agent in the liquid medium may be necessary.

In certain embodiments, the microorganisms can be fermented in a pH range from about 2.0 to about 10.0 and, more specifically, at a pH range of from about 3.0 to about 7.0 (by manually or automatically adjusting pH using bases, acids, and buffers; e.g., HCl, KOH, NaOH, H₂SO₄, and/or H₃PO₄). The invention can also be practiced outside of this pH range.

The fermentation can start at a first pH (e.g., a pH of 4.0 to 4.5) and later change to a second pH (e.g., a pH of 3.2-3.5) for the remainder of the process to help avoid contamination as well as to produce other desirable results (the first pH can be either higher or lower than the second pH). In some embodiments, pH is adjusted from a first pH to a second pH after a desired accumulation of biomass is achieved, for example, from 0 hours to 200 hours after the start of fermentation, more specifically from 12 to 120 hours after, more specifically from 24 to 72 hours after.

In one embodiment, the moisture level of the culture medium should be suitable for the microorganism of interest. In a further embodiment, the moisture level may range from 20% to 90%, preferably, from 30 to 80%, more preferably, from 40 to 60%. The cultivation processes of the present invention can be anaerobic, aerobic, or a combination thereof. Preferably, the process is aerobic, keeping the dissolved oxygen concentration above 10 or 15% of saturation during fermentation, but within 20% in some embodiments, or within 30% in some embodiments.

Advantageously, the system/apparatus provides easy oxygenation of the growing culture with, for example, slow motion of air to remove low-oxygen containing air and introduction of oxygenated air. The oxygenated air may be ambient air supplemented periodically, such as daily.

Additionally, antifoaming agents can also be added to the system/apparatus to prevent or inhibit the formation and/or accumulation of foam during cultivation and fermentation.

In one embodiment, the microbe-based composition does not need to be further processed after fermentation (e.g., yeast, metabolites, and remaining carbon sources do not need to be separated from the sophorolipids). The physical properties of the final product (e.g., viscosity, density, etc.) can also be adjusted using various chemicals and materials that are known in the art.

In one embodiment, the culture medium used in the subject system/apparatus, may contain supplemental nutrients for the microorganism. Typically, these include carbon sources, proteins, fats, or lipids, nitrogen sources, trace elements, and/or growth factors (e.g., vitamins, pH regulators). It will be apparent to one of skill in the art that nutrient concentration, moisture content, pH, and the like may be modulated to optimize growth for a particular microbe.

The lipid source can include oils or fats of plant or animal origin which contain free fatty acids or their salts or their esters, including triglycerides. Examples of fatty acids include, but are not limited to, free and esterified fatty acids containing from 16 to 18 carbon atoms, hydrophobic carbon sources, palm oil, animal fats, coconut oil, oleic acid, soybean oil, sunflower oil, canola oil, stearic and palmitic acid.

The culture medium of the subject system/apparatus can further comprise a carbon source. The carbon source is typically a carbohydrate, such as glucose, xylose, sucrose, lactose, fructose, trehalose, galactose, mannose, mannitol, sorbose, ribose, and maltose; organic acids such as acetic acid, fumaric acid, citric acid, propionic acid, malic acid, malonic acid, and pyruvic acid; alcohols such as ethanol, propanol, butanol, pentanol, hexanol, erythritol, isobutanol, xylitol, and glycerol; fats and oils such as canola oil, soybean oil, rice bran oil, olive oil, corn oil, sesame oil, and linseed oil; etc. Other carbon sources can include arbutin, raffinose, gluconate, citrate, molasses, hydrolyzed starch, potato extract, corn syrup, and hydrolyzed cellulosic material. The above carbon sources may be used independently or in a combination of two or more.

In one embodiment, growth factors and trace nutrients for microorganisms are included in the medium of the system. This is particularly preferred when growing microbes that are incapable of producing all of the vitamins they require. Inorganic nutrients, including trace elements such as iron, zinc, potassium, calcium copper, manganese, molybdenum and cobalt; phosphorous, such as from phosphates; and other growth stimulating components can be included in the culture medium of the subject systems. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included, e.g., L-Alanine.

In one embodiment, inorganic or mineral salts may also be included. Inorganic salts can be, for example, potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, calcium carbonate, sodium carbonate. These inorganic salts may be used independently or in a combination of two or more.

The culture medium of the subject system/apparatus can further comprise a nitrogen source. The nitrogen source can be, for example, in an inorganic form such as potassium nitrate, ammonium nitrate, ammonium sulfate, ammonium phosphate, ammonia, urea, and ammonium chloride, or an organic form such as proteins, amino acids, yeast extracts, yeast autolysates, corn peptone, casein hydrolysate, and soybean protein. These nitrogen sources may be used independently or in a combination of two or more.

The microbes can be grown in planktonic form or as biofilm. In the case of biofilm, the vessel may have within it a substrate upon which the microbes can be grown in a biofilm state. The system/apparatus may also have, for example, the capacity to apply stimuli (such as shear stress) that encourages and/or improves the biofilm growth characteristics.

### Preparation of Microbe-Based Products

The microbe-based products of the present invention include products comprising the microbes and/or microbial growth by-products and optionally, the growth medium and/or additional ingredients such as, for example, water, carriers, adjuvants, nutrients, viscosity modifiers, and other active agents.

One microbe-based product of the present invention is simply the fermentation medium containing the microorganism and/or the microbial growth by-products produced by the microorganism and/or any residual nutrients. The product of fermentation may be used directly without extraction or purification. If desired, extraction and purification can be easily achieved using standard extraction methods or techniques known to those skilled in the art.

The microorganisms in the microbe-based products may be in an active or inactive form and/or in the form of vegetative cells, spores, mycelia, conidia and/or any form of microbial propagule. The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

The microbes and/or medium resulting from the microbial growth can be removed from the growth vessel and transferred via, for example, piping for immediate use.

In other embodiments, the composition (microbes, medium, or microbes and medium) can be placed in containers of appropriate size, taking into consideration, for example, the intended use, the contemplated method of application, the size of the fermentation tank, and any mode of transportation from microbe growth facility to the location of use. Thus, the containers into which the microbe-based composition is placed may be, for example, from 1 gallon to 1,000 gallons or more. In other embodiments the containers are 2 gallons, 5 gallons, 25 gallons, or larger.

Upon harvesting the microbe-based composition from the growth vessels, further components can be added as the harvested product is placed into containers and/or piped (or otherwise transported for use). The additives can be, for example, buffers, carriers, other microbe-based compositions produced at the same or different facility, viscosity modifiers, preservatives, nutrients for microbe growth, nutrients for plant growth, tracking agents, pesticides, herbicides, animal feed, food products and other ingredients specific for an intended use.

Advantageously, in accordance with the present invention, the microbe-based product may comprise broth in which the microbes were grown. The product may be, for example, at least, by weight, 1%, 5%, 10%, 25%, 50%, 75%, or 100% broth. The amount of biomass in the product, by weight, may be, for example, anywhere from 0% to 100% inclusive of all percentages therebetween.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C. On the other hand, a biosurfactant composition can typically be stored at ambient temperatures.

The microbe-based products of the present invention may be, for example, microbial inoculants, biopesticides, nutrient sources, remediation agents, health products, and/or biosurfactants.

In one embodiment, the fermentation products (e.g., microorganisms and/or metabolites) obtained after the cultivation process are typically of high commercial value. Those products containing microorganisms have enhanced nutrient content than those products deficient in the microorganisms. The microorganisms may be present in the cultivation system/apparatus, the cultivation broth and/or cultivation biomass. The cultivation broth and/or biomass may be dried (e.g., spray-dried), to produce the products of interest.

In one embodiment, the cultivation products may be prepared as a spray-dried biomass product. The biomass may be separated by known methods, such as centrifugation, filtration, separation, decanting, a combination of separation and decanting, ultrafiltration or microfiltration. The biomass cultivation products may be further treated to facilitate rumen bypass. The biomass product may be separated from the cultivation medium, spray-dried, and optionally treated to modulate rumen bypass, and added to feed as a nutritional source.

In one embodiment, the cultivation products may be used as an animal feed or as food supplement for humans. The cultivation products may be rich in at least one or more of fats, fatty acids, lipids such as phospholipid, vitamins, essential amino acids, peptides, proteins, carbohydrates, sterols, enzymes, and trace minerals such as, iron, copper, zinc, manganese, cobalt, iodine, selenium, molybdenum, nickel, fluorine, vanadium, tin and silicon. The peptides may contain at least one essential amino acid.

In other embodiments, the essential amino acids are encapsulated inside a subject modified microorganism used in a cultivation reaction. The essential amino acids are contained in heterologous polypeptides expressed by the microorganism. Where desired, the heterologous peptides are expressed and stored in the inclusion bodies in a suitable microorganism (e.g., fungi).

In one embodiment, the cultivation products have a high nutritional content. As a result, a higher percentage of the cultivation products may be used in a complete animal feed. In one embodiment, the feed composition comprises the modified cultivation products ranging from 15% of the feed to 100% of the feed.

The present invention further provides materials and methods for the production of biomass (e.g., viable cellular material), extracellular metabolites (e.g., both small and large molecules), and/or intracellular components (e.g., enzymes and other proteins). The microbes and microbial growth by-products of the present invention can also be used for the transformation of a substrate, such as an ore, wherein the transformed substrate is the product.

The present invention further provides microbe-based products, as well as uses for these products to achieve beneficial results in many settings including, for example, improved bioremediation, mining, and oil and gas production; waste disposal and treatment; enhanced health of livestock and other animals; and enhanced health and productivity of plants by applying one or more of the microbe-based products.

In specific embodiments, the system/apparatus of the present invention provide science-based solutions that improve agricultural productivity by, for example, promoting crop vitality; enhancing crop yields; enhancing plant immune responses; enhancing insect, pest and disease resistance; controlling insects, nematodes, diseases and weeds; improving plant nutrition; improving the nutritional content of agricultural and forestry and pasture soils; and promoting improved and more efficient water use.

In one embodiment, the present invention provides a method of improving plant health and/or increasing crop yield by applying the composition disclosed herein to soil, seed, or plant parts. In another embodiment, the present invention provides a method of increasing crop or plant yield comprising multiple applications of the composition described herein.

Advantageously, the method can effectively control nematodes, and the corresponding diseases caused by pests while a yield increase is achieved and side effects and additional costs are avoided.

In another embodiment, the method for producing microbial growth by-products may further comprise steps of concentrating and purifying the by-product of interest.

In one embodiment, the present invention further provides a composition comprising at least one type of microorganism and/or at least one microbial growth by-product produced by said microorganism. The microorganisms in the composition may be in an active or inactive form and/or in the form of vegetative cells, spores, mycelia, conidia and/or any form of microbial propagule. The composition may or may not comprise the growth matrix in which the microbes were grown. The composition may also be in a dried form or a liquid form.

In one embodiment, the composition is suitable for agriculture. For example, the composition can be used to treat soil, plants, and seeds. The composition may also be used as a pesticide.

In one embodiment, the present invention further provides customizations to the materials and methods according to the local needs. For example, the method for cultivation of microorganisms may be used to grow those microorganisms located in the local soil or at a specific oil well or site of pollution. In specific embodiments, local soils may be used as the solid substrates in the cultivation method for providing a native growth environment. Advantageously, these microorganisms can be beneficial and more adaptable to local needs.

The cultivation method according to the present invention not only substantially increases the yield of microbial products per unit of nutrient medium but also improves the simplicity of the production operation. Furthermore, the cultivation process can eliminate or reduce the need to concentrate microorganisms after finalizing fermentation.

Advantageously, the method does not require complicated equipment or high energy consumption, and thus reduces the capital and labor costs of producing microorganisms and their metabolites on a large scale.

### Microbial Growth By-Products

The methods and system/apparatus of the present invention can be used to produce useful microbial growth by-products such as, for example, biosurfactants, enzymes, acids, biopolymers, solvents, and/or other microbial metabolites. In specific embodiments, the growth by-product is a biosurfactant. Even more specifically, the growth by-product can be SLP.

Biosurfactants are a structurally diverse group of surface-active substances produced by microorganisms. Biosurfactants are biodegradable and can be easily and cheaply produced using selected organisms on renewable substrates. Most biosurfactant-producing organisms produce biosurfactants in response to the presence of a hydrocarbon source (e.g., oils, sugar, glycerol, etc.) in the growing media. Other media components such as concentration of iron can also affect biosurfactant production significantly.

All biosurfactants are amphiphiles. They consist of two parts: a polar (hydrophilic) moiety and non-polar (hydrophobic) group. Due to their amphiphilic structure, biosurfactants increase the surface area of hydrophobic water-insoluble substances, increase the water bioavailability of such substances, and change the properties of bacterial cell surfaces.

Biosurfactants include low molecular weight glycolipids (e.g., rhamnolipids, sophorolipids, mannosylerythritol lipids), lipopeptides (e.g., surfactin), flavolipids, phospholipids, and high molecular weight polymers such as lipoproteins, lipopolysaccharide-protein complexes, and polysaccharide-protein-fatty acid complexes. The common lipophilic moiety of a biosurfactant molecule is the hydrocarbon chain of a fatty acid, whereas the hydrophilic part is formed by ester or alcohol groups of neutral lipids, by the carboxylate group of fatty acids or amino acids (or peptides), organic acid in the case of flavolipids, or, in the case of glycolipids, by the carbohydrate.

Microbial biosurfactants are produced by a variety of microorganisms such as bacteria, fungi, and yeasts. Exemplary biosurfactant-producing microorganisms include Pseudomonas species (P. aeruginosa, P. putida, P. florescens, P. fragi, P. syringae); Flavobacterium spp.; Bacillus spp. (B. subtilis, B. pumillus, B. cereus, B. licheniformis); Wickerhamomyces spp., Candida spp. (C. albicans, C. rugosa, C. tropicalis, C. lipolytica, C. torulopsis); Rhodococcus spp.; Arthrobacter spp.; Campylobacter spp.; Cornybacterium spp.; Pichia spp.; Starmerella spp.; and so on. The biosurfactants may be obtained by fermentation processes known in the art.

Other microbial strains including, for example, other fungal strains capable of accumulating significant amounts of glycolipid-biosurfactants, for example, and/or bacterial strains capable of accumulating significant amounts of, surfactin, for example, can be used in accordance with the present invention. Other metabolites useful according to the present invention include mannoprotein, beta-glucan and other biochemicals that have bio-emulsifying and surface/interfacial tension-reducing properties.

In many embodiments, the subject system is used to produce SLPs on a large scale.

Sophorolipids are glycolipid biosurfactants produced by, for example, various yeasts of the Starmerella clade. Among yeasts of the Starmerella clade that have been examined, the greatest yield of sophorolipids has been reported from Candida apicola and Starmerella bombicola. SLPs consist of a disaccharide sophorose linked to long chain hydroxy fatty acids. These SLPs are a partially acetylated 2-O-β-D-glucopyranosyl-D-glucopyranose unit attached β-glycosidically to 17-L-hydroxyoctadecanoic or 17-L-hydroxy-Δ9-octadecenoic acid. The hydroxy fatty acid is generally 16 or 18 carbon atoms, and may contain one or more unsaturated bonds. The fatty acid carboxyl group can be free (acidic or open form) or internally esterified at the 4″-position (lactone form).

Due to the structure and composition of SLPs, these biosurfactants have excellent surface and interfacial tension reduction properties, as well as other beneficial biochemical properties, which can be useful in applications such as large scale industrial and agriculture uses, and in other fields, including but not limited to cosmetics, household products, and health, medical and pharmaceutical fields.

Biosurfactants accumulate at interfaces, thus reducing interfacial tension and leading to the formation of aggregated micellular structures in solution. Safe, effective microbial biosurfactants reduce the surface and interfacial tensions between the molecules of liquids, solids, and gases. The ability of biosurfactants to form pores and destabilize biological membranes permits their use as antibacterial, antifungal, and hemolytic agents. Combined with the characteristics of low toxicity and biodegradability, biosurfactants are advantageous for use in the oil and gas industry for a wide variety of petroleum industry applications, such as microbially enhanced oil recovery. These applications include, but are not limited to, enhancement of crude oil recovery from an oil-containing formation; stimulation of oil and gas wells (to improve the flow of oil into the well bore); removal of contaminants and/or obstructions such as paraffins, asphaltenes and scale from equipment such as rods, tubing, liners, tanks and pumps; prevention of the corrosion of oil and gas production and transportation equipment; reduction of H₂S concentration in crude oil and natural gas; reduction in viscosity of crude oil; upgradation of heavy crude oils and asphaltenes into lighter hydrocarbon fractions; cleaning of tanks, flowlines and pipelines; enhancing the mobility of oil during water flooding though selective and non-selective plugging; and fracturing fluids.

When used in oil and gas applications, the systems/apparatuses of the present invention can be used to lower the cost of microbial-based oilfield compositions and can be used in combination with other chemical enhancers, such as polymers, solvents, fracking sand and beads, emulsifiers, surfactants, and other materials known in the art.

Biosurfactants produced according to the present invention can be used for other, non-oil recovery purposes including, for example, cleaning pipes, reactors, and other machinery or surfaces, as well as pest control, for example, when applied to plants and/or their surrounding environment. Some biosurfactants produced according to the present invention can be used to control pests because they are able to penetrate through pests' tissues and are effective in low amounts without the use of adjuvants. It has been found that at concentrations above the critical micelle concentration, the biosurfactants are able to penetrate more effectively into treated objects.

Pests can be controlled using either the biosurfactant-producing organisms as a biocontrol agent or by the biosurfactants themselves. In addition, pest control can be achieved by the use of specific substrates to support the growth of biosurfactant-producing organisms as well as to produce biosurfactant pesticidal agents. Advantageously, natural biosurfactants are able to inhibit the growth of competing organisms and enhance the growth of the specific biosurfactant-producing organisms.

In addition, these biosurfactants can play important roles in treating animal and human diseases. Animals can be treated by, for example, by dipping or bathing in a biosurfactant solution alone, with or without microbe cell mass, and/or in the presence of other compounds such as copper or zinc.

The compositions produced according to the present invention have advantages over biosurfactants alone due to the use of entire cell culture, including: high concentrations of mannoprotein as a part of yeast cell wall's outer surface (mannoprotein is a highly effective bioemulsifier capable of reaching up to an 80% emulsification index); the presence of the biopolymer beta-glucan (an emulsifier) in yeast cell walls; the presence of sophorolipids in the culture, which is a powerful biosurfactant capable of reducing both surface and interfacial tension; and the presence of metabolites (e.g., lactic acid, ethanol, etc.) in the culture. These compositions can, among many other uses, act as biosurfactants and can have surface/interfacial tension-reducing properties.

Cultivation of microbial biosurfactants according to the prior art is a complex, time and resource consuming, process that requires multiple stages. The present invention provides equipment, apparatuses, methods and systems that simplify and reduce the cost of this process. The present invention also provides novel compositions and uses of these compositions.

Although SLPs have been discussed extensively herein, the methods and apparatuses of the present invention can also be used to produce other biosurfactants, including surfactin, rhamnolipids (RLPs), and/or mannosylerythritol lipids (MELs).

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1

### Two-Vessel Apparatus for SLP Production

FIG. 1 shows an image of the two-vessel reactor apparatus. The apparatus includes a fermentation vessel and a collector vessel connected to the fermentation vessel. The collector vessel serves as a collector of a portion of the culture from the fermentation vessel and for precipitation of accumulated SLP, which can then be removed/collected from the collection vessel. The vessels are positioned relatively close to each other to minimize the length of pipe between them. Each vessel is a metal vessel and is supported by a metal frame. such that it is easier to access and to view the inside through the sight glass. Large amounts of SLP can be produced by this reactor apparatus.

The reactor apparatus is for non-stop/continuous fermentation. In the fermentation vessel, the culture is moving and the cells and any SLP present do not settle. In the collector vessel, the cells and medium are static. A Chemostat (chemical environment is static) is a vessel where fresh medium is continuously added, while liquid containing leftover nutrients, metabolic end products, and microbes is continuously removed at the same rate to keep the culture volume constant. The collector vessel is static, meaning that volume of culture (and any other additives) is added at the same rate as volume of products and leftover culture (and any other components removed) is removed. One third or approximately one third of the culture is SLP, which settles to the bottom. SLP precipitates when there is no disturbance. Initially, fermentation is allowed to proceed for four or five days to allow for SLP production. The SLP is then sampled from the collector vessel, and the concentration/amount is measured. Then, if enough SLP has accumulated, SLP can be collected daily. A pump (e.g., a peristaltic pump) or simple gravity can be used to drain/collect the product from the collector vessel. The sight glass can be used to observe SLP accumulation.

The reactor apparatus is self-sterilizing. Once set up and cleaned initially, it does not have to be cleaned. SLP provides self-treatment of sepsis, even at low concentrations, thereby saving the time and cost of sterilization.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety, including all figures and tables, to the extent they are not inconsistent with the explicit teachings of this specification.

1. An apparatus for production of sophorolipids (SLP), the apparatus comprising:
a fermentation vessel;
a collector vessel connected directly to the fermentation vessel;
a means for collecting SLP from the collector vessel; and
a pump for pumping culture from the collector vessel to the fermentation vessel after SLP has been collected from the collector vessel.

2. (canceled)

3. The apparatus of claim 1, wherein the fermentation vessel comprises an impeller.

4. The apparatus of claim 1, wherein the collector vessel comprises an impeller.

5. The apparatus of claim 1, wherein the fermentation vessel comprises a jacket.

6. The apparatus of claim 1, wherein the collector vessel comprises a jacket.

7. The apparatus of claim 1, wherein the means for collecting SLP from the collector vessel is a drain portion at a lower section of the collector vessel, such that gravity can be used to collect precipitated SLP from the lower section of the collector vessel when the drain portion is opened.

8. The apparatus of claim 1, wherein the means for collecting SLP from the collector vessel is a peristaltic pump configured to pump precipitated SLP from a lower section of the collector vessel.

9-10. (canceled)

11. The apparatus of claim 1, further comprising a carbon source vessel connected directly to at least one of the fermentation vessel and the collector vessel, the carbon source vessel containing a sugar source and a fatty acid source.

12. (canceled)

13. The apparatus of claim 11, further comprising:
a first carbon source vessel connected directly to at least one of the fermentation vessel and the collector vessel, the first carbon source vessel containing a sugar source; and
a second carbon source vessel connected directly to at least one of the fermentation vessel and the collector vessel, the second carbon source vessel containing a fatty acid source.

14-19. (canceled)

20. The apparatus of claim 1, wherein the collector vessel is connected directly to the fermentation vessel via at least one pipe.

21. (canceled)

22. The apparatus of claim 1, wherein the apparatus comprises wheels and handles for maneuvering the apparatus by hand.

23. A method for production of sophorolipids (SLP), wherein said method comprises:
adding a culture medium and a microorganism to the fermentation vessel of the apparatus of claim 1;
allowing fermentation to proceed for a period of time to allow for SLP production; and
collecting precipitated SLP from the collector vessel.

24. The method of claim 23, wherein the microorganism is a yeast Starmerella bombicola.

25-29. (canceled)

30. The method of claim 23, wherein, after each collection of precipitated SLP from the collector vessel, the culture from the collector vessel is pumped back to the fermentation vessel using the pump.

31. The method of claim 30, wherein, before the culture from the collector vessel is pumped back to the fermentation vessel, a sugar source and a fatty acid source are added to the collector vessel.

32-35. (canceled)

36. The method of claim 23, wherein, at the time of the each collection of SLP from the collector vessel, approximately one third (by volume) of the culture in the collector vessel is SLP.

37. The method of claim 23, wherein the apparatus is sterilized prior to cultivating the microorganism and wherein the sterilization comprises:
washing internal surfaces of the fermentation vessel and the collector vessel with a disinfectant:
fogging the inside of thefermentation vessel and the collector vessel with a 3% hydrogen peroxide solution; and
steaming the inside of the fermentation vessel and the collector vessel with water at a temperature of 105 ° C. to 110 ° C.

38-46. (canceled)
